(19) United States
(12) Patent Application Publication
(10) Pub. No.: US 2025/0218544 A1
(43) Pub. Date: Jul. 3, 2025

(54) METHOD FOR FINDING PEPTIDE LINKERS BETWEEN DIFFERENT PEPTIDES

(71) Applicant: Acer Incorporated, New Taipei City (TW)

(72) Inventors: Chih-Wei Tu, New Taipei City (TW); Yun-Hsuan Chan, New Taipei City (TW); Ke-Han Pan, New Taipei City (TW); Tsung-Hsien Tsai, New Taipei City (TW); Li-Tzu Yeh, New Taipei City (TW); Jhe-Jhih Lin, New Taipei City (TW)

(73) Assignee: Acer Incorporated, New Taipei City (TW)

(21) Appl. No.: 18/964,718

(22) Filed: Dec. 2, 2024

Related U.S. Application Data
(60) Provisional application No. 63/615,269, filed on Dec. 27, 2023.

(30) Foreign Application Priority Data
Oct. 14, 2024 (TW) 113138936

(51) Int. Cl. G16B 30/20

(52) U.S. Cl. CPC G16B 30/20

(57) ABSTRACT

The invention provides a method for finding peptide linkers between different peptides. The method includes the following steps. A long sequence is composed, which includes of a plurality of different peptides and peptide linkers between the peptides. Based on cleavage site probabilities predicted by Model A, peptide linker combinations are sorted according to a expressions of the peptides and peptide linkers, and the TopN peptide linker combinations are selected. The TopN peptide linker combinations are applied to other models for prediction, the cleavage site probabilities predicted by other models are considered, and a ranking of peptide linker combinations is generated. Commonly selected peptide linkers are combined and the peptide linker combination with the highest weighted average ranking is selected.

### CROSS-REFERENCE TO RELATED APPLICATION

This application claims the priority benefit of U.S. provisional application Ser. No. 63/615,269, filed on Dec. 27, 2023, and Taiwan application serial no. 113138936, filed on Oct. 14, 2024. The entirety of each of the above-mentioned patent applications is hereby incorporated by reference herein and made a part of this specification.

### BACKGROUND

### Technical Field

The invention relates to a method for finding peptide linkers, and particularly to a method for finding peptide linkers between different peptides.

### Description of Related Art

With the advancement of next-generation gene sequencing and artificial intelligence, personalized cancer vaccines have become one of the means for cancer treatment. The vector template structure of personalized cancer vaccines includes: tumor neoantigen priority sorting and selection, followed by connecting peptides with high priority through peptide linkers.

Among them, peptide linkers, in addition to connecting different possible tumor neoantigens, also play an auxiliary role in assisting the possibility of peptides generating immune response mechanisms. However, the current selection of peptide linkers is mainly based on the characteristics of not inducing an immune response to determine the sequence of peptide linkers. This method ignores the interaction between peptides and linkers, which may prevent peptides from being effectively cleaved to induce downstream immune responses. Therefore, it is necessary to consider the relationship between peptides and peptide linkers, and develop a method to find suitable linkers between different peptides.

Existing technology only uses fixed linkers to connect peptides, without considering that the same linker between different peptides will have different reactions after proteolytic cleavage, leading to incomplete cleavage of peptides and thereby affecting the efficacy of peptides. In addition, not all amino acid combinations can be synthesized, which may result in the inability to synthesize the peptides required for the vaccine with fixed linker sequences. Therefore, it is necessary to design exclusive linkers between each different peptide to enhance the efficacy of the peptides.

### SUMMARY

The invention provides a method for finding peptide linkers between different peptides and the peptide linkers, to enhance the efficacy of the peptides.

The method for finding peptide linkers between different peptides of the invention includes the following steps. A long sequence is composed, which includes a plurality of different peptides and peptide linkers between the peptides. Peptide linker combinations are sorted based on cleavage site probabilities predicted by Model A, according to expressions of the peptides and the peptide linkers, and Top N peptide linker combinations are selected. Based on the peptide linker combination sorting, a sorting table is generated, and rankings are given to peptide linker combinations utilizing a main condition and a second condition. The Top N peptide linker combinations are applied to other models for prediction, the cleavage site probabilities predicted by other models are considered, the peptide linker combinations are sorted based on the expression of peptides and peptide linkers, main condition sorting tables for other models are generated, and the second condition for sorting is utilized to generate peptide linker combination rankings. The combination selection order is at least equal to or better than the number of Model A, and the commonly selected peptide linkers are subjected to further analysis. Commonly selected peptide linkers are combined and the peptide linker combination with the highest weighted average ranking is selected.

In an embodiment of the invention, Model A includes Pepsickle or NetCleave.

In an embodiment of the invention, the main condition includes a quantity of peptides being cleaved and a ranking of cleavage site locations of peptide linkers.

In an embodiment of the invention, the second condition comprises an average probability of peptide linkers being cleaved minus an average probability of peptides being cleaved.

In an embodiment of the invention, selecting the Top N peptide linker combinations includes selecting the Top N peptide linker combinations one by one according to the combination selection order of the main condition sorting table of Model A, and sorting the Top N peptide linker combinations based on second condition to generate peptide linker combination rankings. Then, other models are utilized for prediction, if suitable peptide linkers are selected, the process stops; if not, selections continue in order until peptide linkers are selected.

Based on the above, the invention provides a method for finding peptide linkers between different peptides. The method considers the relationship between peptides and peptide linkers, taking into account that the same linkers may react differently after proteolytic cleavage between different peptides, in order to find suitable linkers between different peptides, thereby enhancing the efficacy of these peptides.

### BRIEF DESCRIPTION OF THE DRAWINGS

FIG. 1 is a flowchart of a method for finding peptide linkers between different peptides according to an embodiment of the invention.

FIG. 2 is a schematic diagram of a long sequence according to an embodiment of the invention.

FIG. 3 is a schematic diagram of a main condition and a second condition according to an embodiment of the invention.

### DESCRIPTION OF THE EMBODIMENTS

The following examples are described in detail with reference to the accompanying drawings, but the provided embodiment s are not intended to limit the scope covered by this disclosure. Furthermore, terms such as “contain”, “include”, “have”, etc. used in the text are open-ended terms, meaning “including but not limited to”.

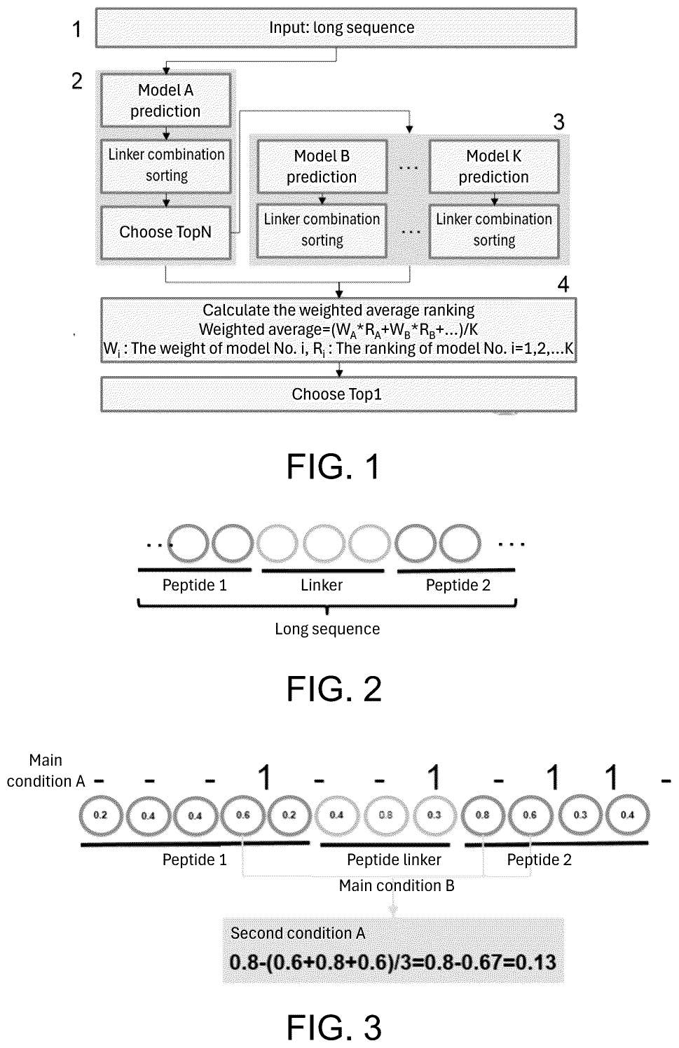

FIG. 1 is a flowchart of a method for finding peptide linkers between different peptides according to an embodiment of the invention. Referring to FIG. 1, the method for finding peptide linkers between different peptides of the invention includes four main parts. The following will introduce these four main parts in order in conjunction with FIG. 1.

FIG. 2 is a schematic diagram of a long sequence according to an embodiment of the invention. Referring to FIG. 1 and FIG. 2, firstly, Part 1 involves composing a long sequence (peptide+peptide linker+peptide), and setting the initial length of the peptide linker=L, resulting in 20 (amino acids)\hat{ }L peptide linker combinations, as shown in FIG. 2 with an example of a peptide linker length of 3.

Referring to FIG. 1, subsequently, Part 2 involves sorting the peptide linker combinations based on the cleavage site probabilities predicted by Model A, according to the expressions of peptides and peptide linkers, and selecting the Top N peptide linker combinations. In this embodiment, Model A is typically the most commonly used model by scholars or the model with the best prediction performance, such as Pepsickle or NetCleave. Based on the peptide linker combination sorting, a sorting table is generated. The peptide linker combinations are ranked according to a main condition and a second condition. For example, Model A may be exemplified by Pepsickle, and other models may be exemplified by NetCleave, but the invention is not limited thereto. The predictions are applied to other K models, so K primary and second condition sorting tables would be generated, which is not limited to two models. If K models are applied, the same screening method is utilized, and the combination selection order must be at least equal to or better than the number of Model A. Additionally, Model A is usually chosen as the best-performing model or the model with a sufficiently large dataset.

Main condition: (assuming the probability of being cleaved is >0.5)
A. The quantity of cleavages in peptide 1 and peptide 2 (the smaller, the better)
B. The ranking of cleavage sites in the peptide linker (defined by the tester, usually expecting the linker to be cleaved in the middle). For example: when the peptide linker length is 3, the location ranking is shown in Table 1 below (a total of 2\hat{ }3-8 rankings).

TABLE 1

| Peptide | | | | |
|---|---|---|---|---|
| linker | | Peptide linker | | |
| position | Peptide linker | second | Peptide linker | |
| sorting | first position | position | third position | Expression |
| 1 | | Cleave | | 010 |
| 2 | Cleave | | | 100 |
| 3 | | | Cleave | 001 |
| 4 | Cleave | Cleave | | 110 |
| 5 | | Cleave | Cleave | 011 |
| 6 | Cleave | | Cleave | 101 |
| 7 | Cleave | Cleave | Cleave | 111 |
| 8 | | | | 000 |

Second condition (assuming the probability of being cleaved is >0.5)
A. The average probability of the peptide linker being cleaved minus the average probability of the peptide being cleaved (the larger, the better; usually hoping for the peptide linker to be cleaved and the peptide not to be cleaved). Referring to the example in FIG. 3, which illustrates the main condition and the second condition according to an embodiment of the invention.

TopN linker combinations are determined. According to the combination selection order in the main condition sorting table of Model A, TopN peptide linker combinations are selected one by one, and these TopN peptide linker combinations are sorted based on the second condition to generate a peptide linker combination ranking. After that, other models are utilized for prediction. If a suitable peptide linker is selected, stop; if not, the selection is continued in order until a peptide linker is selected. As exemplified in Table 2 below: first, based on the combination selection order 1, the Top6 peptide linker combinations will be utilized to predict using models B, C, . . . , K. If no selection is made, then combination selection order 2, 3, 4, . . . are selected, until a peptide linker is selected.

TABLE 2

| | Main | Main | Peptide linker |
|---|---|---|---|
| Combination | condition A | condition B | combination |
| selection | Peptide cleavage | Peptide linker | quantity of |
| order | quantity | cleavage site | Model A |
| 1 | 0 | 010 | 6 |
| 2 | 0 | 100 | 10 |
| 3 | 0 | 001 | 15 |
| 4 | 0 | 110 | 12 |
| 5 | 0 | 011 | . . . |
| 6 | 0 | 101 | . . . |
| 7 | 0 | 111 | . . . |
| 8 | 0 | 000 | . . . |
| 9 | 1 | 010 | . . . |
| . . . | . . . | . . . | . . . |

Referring to FIG. 1, the Part 3 is to apply the TopN peptide linker combinations to other models for prediction, considering the cleavage site probability predicted by other models. The peptide linker combinations are ranked based on the expressions of peptides and peptide linkers, a main condition sorting table for other models is similarly generated, and second conditions for sorting is utilized to produce a peptide linker combination ranking. The combination selection order must be at least equal to or better than the number in Model A, and the commonly selected peptide linkers will be used for further analysis. In this example, the Top6 peptide linkers in combination selection order 1 are predicted using the NetCleave model, and the main condition A, main condition B, and second conditions for these six peptide linkers are obtained. The main condition A is selected: peptide linker with peptide cleavage quantity of 0 (ie., TWG & SWG) and the second condition probability difference is used for sorting, resulting in SWG as rank 1 and TWG as rank 2. (referring to Table 3 below)

TABLE 3

| | Pepsickle (Model A) | | | NetCleave (Model B) | | |
|---|---|---|---|---|---|---|
| | | Main | | | Main | |
| | Main | condition | | Main | condition | |
| | condition | B | | condition | B | |
| | A | Peptide | Second | A | Peptide | Second |
| | Peptide | linker | condition | Peptide | linker | condition |
| Peptide | cleavage | cleavage | Probability | cleavage | cleavage | Probability |
| linker | quantity | site | difference | quantity | site | difference |
| TWR | 0 | 010 | 0.93 | 1 | 010 | 0.13 |
| TWG | 0 | 010 | 0.92 | 0 | 010 | 0.89 |
| EWR | 0 | 010 | 0.92 | 1 | 010 | 0.28 |
| SWG | 0 | 010 | 0.91 | 0 | 010 | 0.91 |
| QYK | 0 | 010 | 0.90 | 1 | 010 | 0.15 |
| EYK | 0 | 010 | 0.90 | 1 | 010 | 0.23 |

Referring to FIG. 1, the Part 4 is to combine the commonly selected peptide linker combinations and select the peptide linker combination with the highest weighted average ranking.

$$Weighted\_Average = ( {W}_{A} * {R}_{A} + {W}_{B} * {R}_{B} + … ) / K$$

Wᵢ: The weight of model No. i, Rᵢ: The ranking of model No. i, i=1, 2, . . . . K

Example explanation: Suppose the peptide linker length is 3, and the weights of Model A and Model B are both 0.5. After screening by combination number 1 of Model A (peptide cleavage quantity=0, peptide linker cleavage site=010), only TWG and SWG meet the main conditions. After calculating the weighted average ranking, the peptide linker TWG will be selected (0.5*1+0.5*2=1.5). (Please refer to Table 4 below)

TABLE 4

| | Pepsickle (Model A) | | | | NetCleave (Model B) | | | | |
|---|---|---|---|---|---|---|---|---|---|
| | | | Main | | | | Main | | |
| | | Main | condition | | | Main | condition | | |
| | | condition | B | | | condition | B | | |
| | | A | Peptide | Second | | A | Peptide | Second | |
| | | Peptide | linker | condition | | Peptide | linker | condition | Weighted |
| Peptide | | cleavage | cleavage | Probability | | cleavage | cleavage | Probability | average |
| linker | Rank | quantity | site | difference | Rank | quantity | site | difference | ranking |
| STWG | 1 | 0 | 010 | 0.92 | 2 | 0 | 010 | 0.89 | 1.5 |
| SWG | 4 | 0 | 010 | 0.91 | 1 | 0 | 010 | 0.91 | 2.5 |

In summary, the invention provides a method for finding peptide linkers between different peptides. The method considers the relationship between peptides and peptide linkers, taking into account that the same linker may react differently to proteolytic cleavage between different peptides, in order to find suitable linkers between different peptides. Therefore, the identified peptide linkers may be utilized to connect tumor neoantigens, designing exclusive linkers for each different peptide pair, thereby enhancing the efficacy of these tumor neoantigens.

What is claimed is:

1. A method for finding peptide linkers between different peptides, comprising:
composing a long sequence, which includes a plurality of different peptides and peptide linkers between the peptides;
sorting peptide linker combinations based on cleavage site probabilities predicted by Model A, according to expressions of the peptides and the peptide linkers, and selecting Top N peptide linker combinations;
generating a sorting table based on the peptide linker combination sorting, and utilizing a main condition and a second condition to rank the peptide linker combinations;
applying the Top N peptide linker combinations to other models for prediction, considering cleavage site probabilities predicted by other models, sorting the peptide linker combinations based on the expressions of the peptides and the peptide linkers, similarly generating main condition sorting tables for other models, and utilizing the second condition for sorting to generate peptide linker combination rankings, wherein a combination selection order is at least equal to or better than a number of Model A, and further analyzing commonly selected peptide linkers; and
selecting commonly selected peptide linker combinations, and selecting the peptide linker combination with a highest weighted average ranking.

2. The method according to claim 1, wherein Model A comprises Pepsickle or NetCleave.

3. The method according to claim 1, wherein the main condition comprises a quantity of peptides being cleaved and a ranking of cleavage site locations of peptide linkers.

4. The method according to claim 1, wherein the second condition comprises an average probability of peptide linkers being cleaved minus an average probability of peptides being cleaved.

5. The method according to claim 1, wherein selecting the Top N peptide linker combinations comprises selecting the Top N peptide linker combinations one by one according to a combination selection order of a main condition sorting table of Model A, and ranking the Top N peptide linker combinations based on the second condition to generate peptide linker combination rankings, then utilizing other models for prediction, stopping if a suitable peptide linker is selected, if not, selecting in order until a peptide linker is selected.
